(19) United States
(12) Patent Application Publication
(10) Pub. No.: US 2009/0247876 A1
(43) Pub. Date: Oct. 1, 2009

(54) LAPAROSCOPIC PROBE GUIDANCE SYSTEM

(76) Inventors: Charles W. Cannon, JR., Jersey Shore, PA (US); David Hull, West Hartford, CT (US)

Correspondence Address: CANTOR COLBURN, LLP, 20 Church Street, 22nd Floor, Hartford, CT, 06103, US

(21) Appl. No.: 12/413,739

(22) Filed: Mar. 30, 2009

Related U.S. Application Data
(60) Provisional application No. 61/040,323, filed on Mar. 28, 2008.

(51) Int. Cl. A61B 8/00

(52) U.S. Cl. 600/461

(57) ABSTRACT

A laparoscopic guidance system is described as attached to an ultrasound transducer or embedded therein for use in surgical procedures for guidance of biopsy and or therapeutic applications whereas a needle is desired to be precisely guided and placed during the procedure. In exemplary embodiments, the guidance system uses both mechanical, electronic laser and imaging systems to combine and project a virtual needle path on an ultrasound image. In exemplary embodiments, the system also provides a projected line for positioning a needle externally or percutaneously to the lesion. The needle may then be inserted and followed in real time to the desired target. The needle and ultrasound probe may be placed and kept in the plane of the image by laser guidance.

### CROSS REFERENCE TO RELATED APPLICATIONS

The present application claims priority to U.S. Provisional Patent Application Ser. No. 61/040,323, filed Mar. 28, 2008, the entire contents of which are specifically incorporated by reference herein.

### BACKGROUND

Today's surgeries are becoming less invasive. Modern medicine is also providing tools that utilize very small needles to destroy diseased tissue. Physicians are now incorporating lore laparoscopic procedures into the operating theater.

Currently, there is no known way of placing needles in a laparoscopic procedure with any assurance that the desired lesion can be accurately targeted. Furthermore, position of trocars utilized during such procedures limits the repositioning of imaging systems. As a result, a desired needle track will often not be possible due to other organs, bone or tissue in the path.

What is needed in the art is a system that ensures accurate targeting of lesions during laparoscopic procedures.

### SUMMARY

The above-described and other problems and disadvantages of the prior art are overcome or alleviated by the present laparoscopic probe guidance system, which system uses one or more of mechanical, electronic laser and imaging components to project a virtual needle path on an ultrasound image. Where desired, a laser guided system will facilitate easier and more accurate placement, offering more alternative paths to place a needle in the desired position.

In exemplary embodiments, the system also provides a projected line for positioning a needle externally or percutaneously to the lesion. The needle may then be inserted and followed in real time to the desired target. The needle and ultrasound probe may be placed and kept in the plane of the image by laser guidance.

An exemplary laparoscopic guidance system may be attached to or embedded in an ultrasound transducer for use in surgical procedures for guidance of biopsy and or therapeutic applications whereas a needle is desired to be precisely guided and placed during the procedure.

### BRIEF DESCRIPTION OF THE DRAWINGS

Referring now to the drawings, wherein like elements are numbered alike in the following FIGURE:

FIG. 1 is an exemplary laser based laparoscopic guidance system and ultrasound probe;

FIG. 2 is an exemplary variation, wherein the prism can be rotated with encoders attached to change the angle of penetration; and

FIG. 3 illustrates an exemplary configuration where as the prism can be rotated and slid along the body of the ultrasound transducer to extend the variable of positioning.

### DETAILED DESCRIPTION OF EXEMPLARY EMBODIMENTS

As is discussed above, the present invention relates to a medical imaging device utilized as a laparoscopic ultrasound unit, comprising of a laparoscopic ultrasound probe and system with an imaging guidance system attached.

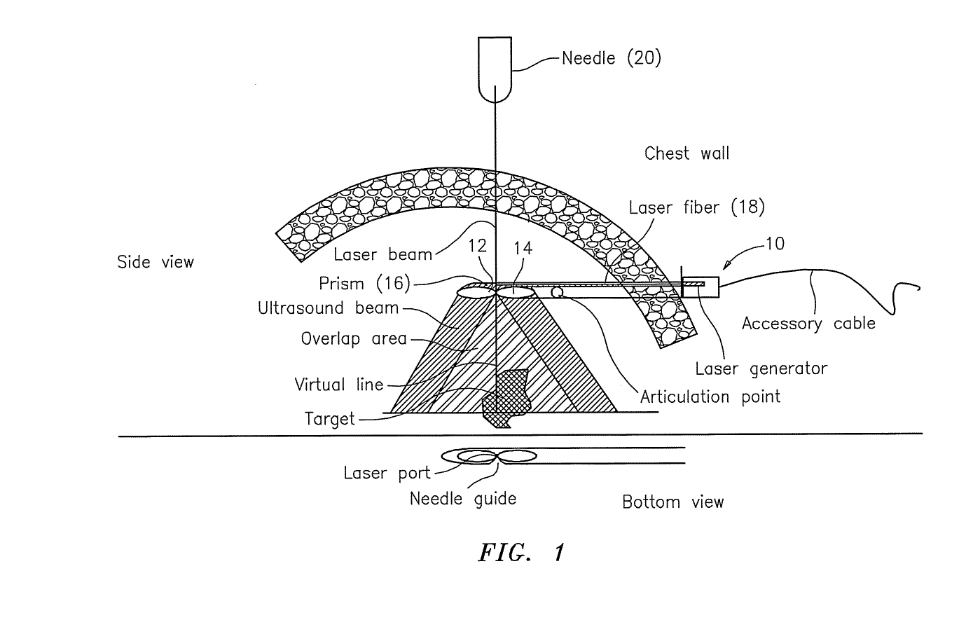

Referring now to FIG. 1, an exemplary system comprises a laparoscopic probe 10 with two convex imagining transducers 12, 14 separated by a small space. A small laser prism 16 is positioned directly between the transducers.

A laser beam is generated in the handle of the transducer and transmitted via fiber optic cable 18 to the prism. The beam then is refracted and sent out perpendicular to the ultrasound transducer head. This beam is then reflected as a dot on the abdominal wall.

A line is superimposed on the image, which line is an extrapolation of the laser. This line is the intended path of the needle 20. With two points established, the dot from the reflection of the laser pointer on the abdominal wall and the recess in the laparoscopic probe the needle path can then be adjusted to optimum location. Once this has been accomplished a needle can be passed and observed real time visually and on the ultrasound.

The illustrated, exemplary ultrasound of FIG. 1 has two transducers to allow for a broader view and space for the needle to pass.

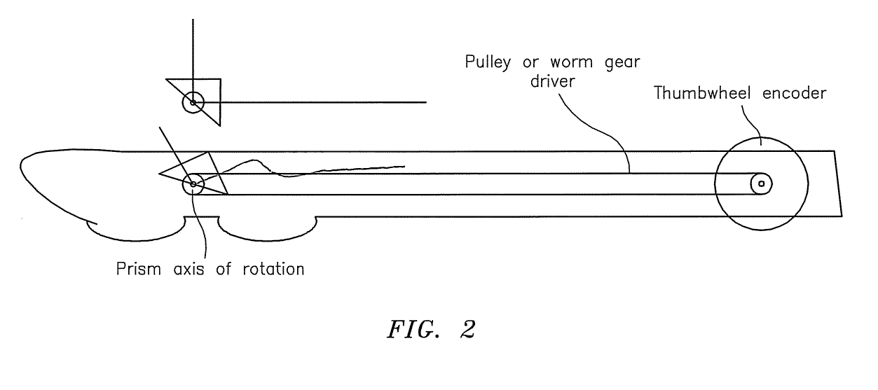

Referring now to FIG. 2, another exemplary variation is illustrated, wherein the prism can be rotated with encoders attached to change the angle of penetration.

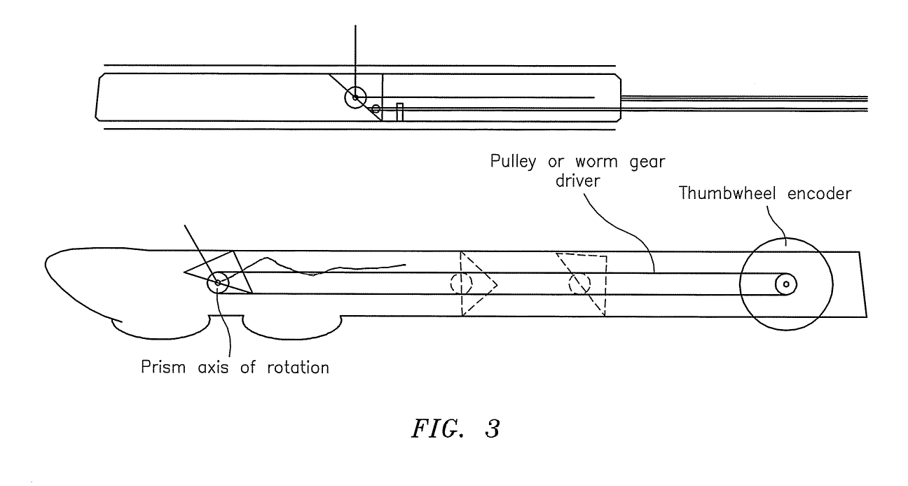

FIG. 3 shows the unit in a configuration where as the prism can be rotated and slid along the body of the ultrasound transducer to extend the variable of positioning.

Thumb screws in the handle may be connected to transducers and mechanically connected to the prism to encode position and angle of the prism.

In alternate embodiments, the prism is eliminated, and the laser is attached directly to the transducer. In exemplary embodiments, single crystal transducers may be used with virtual targeting projected from crystal ends or offsets within the crystal.

It will be apparent to those skilled in the art that, while exemplary embodiments have been shown and described, various modifications and variations can be made to the laparoscopic probe guidance system disclosed herein without departing from the spirit or scope of the invention. Accordingly, it is to be understood that the various embodiments have been described by way of illustration and not limitation.

What is claimed is:

1. A laparoscopic guidance device, comprising:
a laparoscopic ultrasound probe; and
a guidance positioning device attached to or embedded into said laparoscoic ultrasound probe.

2. The laparoscopic guidance device of claim 1, wherein the guidance positioning device is a laser guided positioning system.

3. The laparoscopic guidance device of claim 2, wherein a laser is configured with a pointer to project a start and end point for needle placement and superimpose the target line onto a live (real time) ultrasound image.

4. The laparoscopic guidance device of claim 3, wherein the system is configured such that the path of the needle will be superimposed through on the ultrasound image.

5. The laparoscopic guidance device of claim 1, wherein the guidance positioning device can be both articulated and rotated through handle controls.

6. The laparoscopic guidance device of claim 3, wherein the laser guidance system and virtual guidance track can be relocated to any area along the shaft of the ultrasound probe.

7. The laparoscopic guidance device of claim 6, wherein the angle of approach and position can be adjusted through thumb controls on the Ultrasound probe.

8. The laparoscopic guidance device of claim 1, wherein encoders placed at key articulation points are configured to transmit the position and information transmitted real time to the computer to give constant and real time positioning control of the target path.
